(19) United States
(12) Patent Application Publication
(10) Pub. No.: US 2026/0085952 A1
(43) Pub. Date: Mar. 26, 2026

(54) CONNECTED MONITORING DEVICE

(71) Applicant: Cole-Parmer Instrument Company LLC, Vernon Hills, IL (US)

(72) Inventors: Cameron K. VREELAND, Vernon Hills, IL (US); Boris BASS, Vernon Hills, IL (US); Nathaniel D. KRAFT, Vernon Hills, IL (US); Ashish PATEL, Vernon Hills, IL (US); Chande ESHANI, Vernon Hills, IL (US); Annas BAIDANI, Vernon Hills, IL (US)

(21) Appl. No.: 19/114,245

(22) PCT Filed: Sep. 21, 2023
(86) PCT No.: PCT/US2023/074736

§ 371 Date: Mar. 21, 2025

Related U.S. Application Data
(60) Provisional application No. 63/408,818, filed on Sep. 21, 2022.

(51) Int. Cl. G01D 18/00

(52) U.S. Cl. CPC G01D 18/002

(57) ABSTRACT

Aspects are provided for a connected monitoring device for monitoring and reporting measurements. The monitoring device includes a plurality of sockets, each socket configured to receive a smart probe including a memory storing calibration information for the smart probe. The monitoring device includes a touch screen display configured to display a current measurement based on a received input signal from the smart probe and the calibration information. The monitoring device includes a wireless modem configured to periodically transmit one or more measurements to a remote server via a wireless network.

### CROSS-REFERENCE TO RELATED APPLICATIONS

This application claims priority to U.S. Provisional Application No. 63/408,818 titled “CONNECTED MONITORING DEVICE,” filed Sep. 21, 2022, which is assigned to the assignee hereof, and incorporated herein by reference in its entirety.

### TECHNICAL FIELD

Aspects of the present disclosure generally relate to monitoring devices for observing and recording measurements, and more particularly to a connected monitoring device.

### Introduction

Scientific and industrial processes may be performed under controlled conditions to provide consistency and reproducibility. Some processes may extend for long periods of time during which a technician may be unable to constantly monitor the controlled conditions. Monitoring devices may be used to monitor the controlled conditions for extended periods of time.

Monitoring devices in operation for an extended period of time may face issues with calibration. Over time a probe of a monitoring device may drift from an initial calibrated level such that the accuracy of a measurement may no longer be within tolerances of a protocol. Recalibrating a monitoring device may pose technical challenges to a process because the monitoring device typically needs to be removed from the monitored process to be recalibrated. A gap in the monitoring may lead to an incomplete record for a process.

In view of the foregoing, there is a need for improvements to monitoring devices.

### SUMMARY

The following presents a simplified summary of one or more aspects of the present disclosure in order to provide a basic understanding of such aspects. This summary is not an extensive overview of all contemplated aspects, and is intended to neither identify key or critical elements of all aspects, nor delineate the scope of any or all aspects. Its purpose is to present some concepts of one or more aspects in a simplified form as a prelude to the more detailed description that is presented later.

In some aspects, the techniques described herein relate to a monitoring device including: a plurality of sockets, each socket configured to receive a smart probe including a memory storing calibration information for the smart probe; a touch screen display configured to display a current measurement based on a received input signal from the smart probe and the calibration information; and a wireless modem configured to periodically transmit one or more measurements to a remote server via a wireless network.

In some aspects, the techniques described herein relate to a method of operating a monitoring device, including: reading, from a memory of a smart probe connected to a socket of the monitoring device, calibration information for the smart probe; receiving an input signal from the smart probe, the input signal indicative of a current measurement; determining a value of the current measurement based on the input signal and the calibration information; displaying the value of the current measurement on a display; periodically sampling the current measurement to a local record; and periodically transmitting a portion of the local record to a remote server via a wireless network.

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

### BRIEF DESCRIPTION OF THE DRAWINGS

FIG. 1A is a front perspective view of an example monitoring device, according to an aspect of the disclosure.

FIG. 1B is a rear perspective view of the example monitoring device for FIG. 1A, according to an aspect of the disclosure.

FIG. 1C is an exploded view of a button on the example monitoring device of FIG. 1A, according to an aspect of the disclosure.

FIG. 2A is a perspective view of an example connector of a smart probe, according to an aspect of the disclosure.

FIG. 2B is a perspective view of internal components of the example connector of the smart probe of FIG. 2A, according to an aspect of the disclosure.

FIG. 3 is a block diagram of an example monitoring device, according to an aspect of the disclosure.

FIG. 4 is a message diagram illustrating examples of messages for operating a monitoring device, according to an aspect of the disclosure.

FIG. 5 is a flow diagram showing an example method of operating a monitoring device, according to an aspect of the disclosure.

FIG. 6 is a flow diagram showing additional optional actions for operating a monitoring device in a battery powered mode, according to an aspect of the disclosure.

FIG. 7 is a flow diagram showing additional optional actions for replacing a smart probe, according to an aspect of the disclosure.

FIG. 8 is a flow diagram showing additional optional for monitoring a temperature controlled storage, according to an aspect of the disclosure.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known components are shown in block diagram form in order to avoid obscuring such concepts.

Calibration of a monitoring device is typically completed in a laboratory, where readings of probe are compared against a standard to determine whether the probe is providing a correct reading. In some cases, calibration is merely a verification of the unit under test. Once a unit is deployed in the field, the unit may not be recalibrated for an extended period of time. In some cases, a unit may be associated with an expiration time when the initial calibration is no longer valid, or an error may be detected, for example, via comparison to a second instrument.

Calibration issues may present problems for scientific and industrial processes that are performed over a long time period. For example, if a calibration expires during a process, a record of the controlled conditions may be questionable. If a monitoring device actually drifts from calibrated performance, the record may be corrupted.

Conventional approaches to recalibration of a monitoring device may not preserve a complete record. For example, if a monitoring device needs to be recalibrated, the monitoring device may be removed from the controlled conditions and send to a laboratory for calibration. There may be a gap in the record while the device is absent. Even if a second device is used to monitor the conditions during the gap, there may be inconsistencies in the record.

In an aspect, the present application provides a monitoring device with sockets for interchangeable smart probes. A smart probe is a sensing device that includes a memory that stores calibration information for the smart probe. When a smart probe is inserted into a socket, the monitoring device may read the calibration information from the smart probe. The monitoring device may determine a current measurement based on the calibration information and an input signal from the smart probe. The monitoring device may display the current measurement and also periodically sample the current measurement to generate a record of the measurement. The record of the measurement is stored locally on the monitoring device and periodically transmitted to a server.

In some implementations, the monitoring device and server also provide alarms based on the current measurement and/or record. For example, the monitoring device may be configured with measurement thresholds and/or time thresholds and generate a local and/or remote alert if a condition is satisfied. In some implementations, conditions may be based on two or more sensors to reduce false alarms. For example, a temperature sensor and a door sensor (e.g., a contact sensor or a pressure sensor) may be checked to allow temperature to fluctuate when a door is opened for a short duration, but generate an alarm if the temperature varies greatly or the door remains opened.

In some implementations, the monitoring device may be configured based on a device template. A device identifier may be associated with the device template when the monitoring device is purchased. When the monitoring device connects to the server, the device template is provided to the monitoring device. The monitoring device may be configured based on the device template, for example, with a sampling period, a reporting period, and alarm thresholds. The monitoring device may provide on-screen instructions for connecting and using smart probes according to the device template. The monitoring device may be updated via a batch update to the device template.

In an aspect, the monitoring device of the present disclosure may provide one or more of the following technical effects. The monitoring device may improve accuracy and reliability of a record of measurements by ensuring calibration of smart probes. The interchangeable smart probes may allow a continuous record when a smart probe is removed for recalibration and immediately replaced with a calibrated probe (e.g., within a sampling or reporting period). Additionally, the ease of use of the monitoring device may be improved via automatic configuration using a device template.

Several aspects of a connected monitoring device will now be presented with reference to various apparatus and methods. These apparatus and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more example implementations, the functions described may be implemented in hardware, software, or any combination thereof. If implemented in software, the functions may be stored on or encoded as one or more instructions or code on a computer-readable medium. Computer-readable media includes computer storage media, which may be referred to as non-transitory computer-readable media. Non-transitory computer-readable media excludes transitory signals. Storage media may be any available media that can be accessed by a computer. By way of example, and not limitation, such computer-readable media can include a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

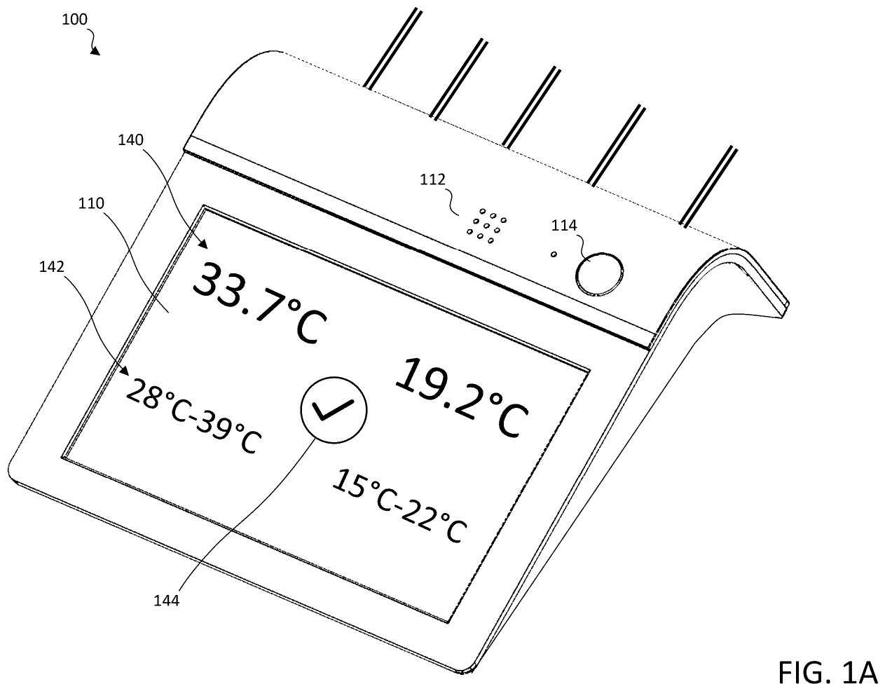

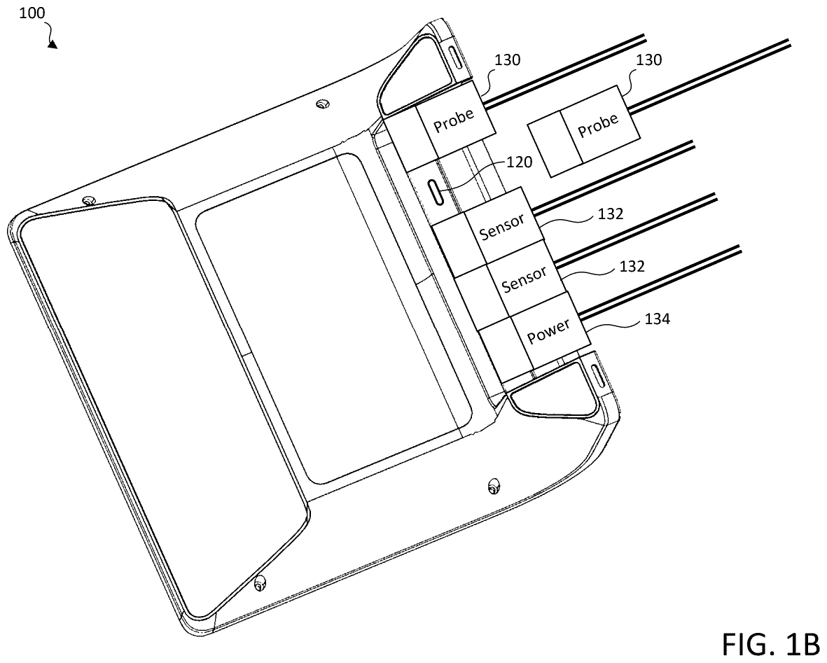

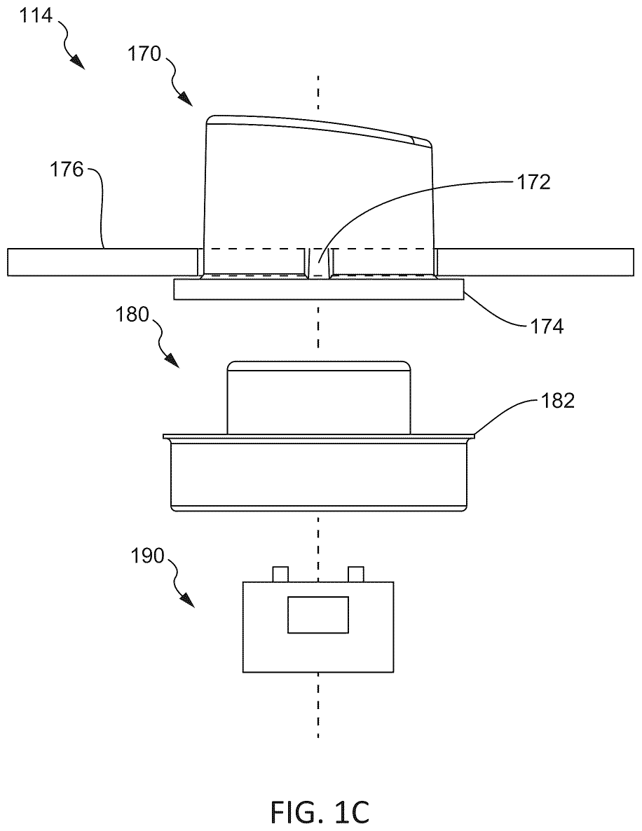

FIG. 1A is front perspective view of an example monitoring device 100. The monitoring device 100 includes a display 110 that is configured to provide a user interface. The display 110 may be, for example, a touch-screen display. The display 110 may present one or more current measurements 140. For example, the illustrated display 110 shows a current measurement 140 for two temperature probes. The display 110 may also show an acceptable range 142 for the current measurement 140. In some implementations, the display 110 may include an indicator 144 that all current measurements are within a corresponding acceptable range 142. Accordingly, a user may observe quickly that there are no alarms associated with the monitoring device. The indicator 144 may change appearance (e.g., color and symbol) when an alarm is triggered.

In an aspect, the display 110 of the monitoring device 100 is angled with respect to a mounting surface. For example, the monitoring device 100 may be mounted to a horizontal or vertical surface. The angle of the display 110 may allow the contents of the display to be read from a distance from various perspectives. For instance, when placed horizontally on a table or counter, the display 110 may be read from a seated or standing height. If mounted to a vertical surface of a storage unit, the monitoring device may be read from both the front and the side of the storage unit.

The monitoring device 100 further includes a speaker 112 and a button 114. The speaker 112 is configured to output audible alarms. The button 114 may control power for the monitoring device 100 and/or the display 110. The button 114 may also activate a configuration mode. For instance, a single press of the button 114 may activate the configuration mode, a double press may turn the display 110 on or off, and a long hold may turn monitoring device on or off. In some implementations, the button 114 may be liquid-proof.

FIG. 1B is rear perspective view of the example monitoring device 100. The monitoring device 100 includes a plurality of sockets 120. Each socket 120 may receive a smart probe 130 or a sensor 132. For instance, a probe 130 may provide a continuous input signal such as an analog signal. A sensor 132 may provide a discrete or binary input signal. In some implementations, a socket 120 may receive either a probe 130 or a sensor 132. In some implementations, the socket 120 may be a female universal serial bus (USB) port such as a USB Type-C connector. The monitoring device 100 may include a power port 134, which may be another USB port.

FIG. 1C is an exploded view of the button 114. The button 114 includes a contact portion 170, a boot 180, and an electric button 190. The contact portion 170 extends through an opening of the housing 176 of the monitoring device 100. The contact portion 170 includes a registration feature 172 that engages a groove on the housing 176 to maintain orientation of the contact portion 170. The boot 180 receives a flange 174 of the contact portion 170. The boot 180 is a flexible material (e.g., rubber) that stretches as the button is pressed to provide resistance. A lip 182 of the boot 180 is sealed to the housing 176 to create a liquid-proof barrier. The boot 180 contacts the electric button 190 to register a press of the button 114.

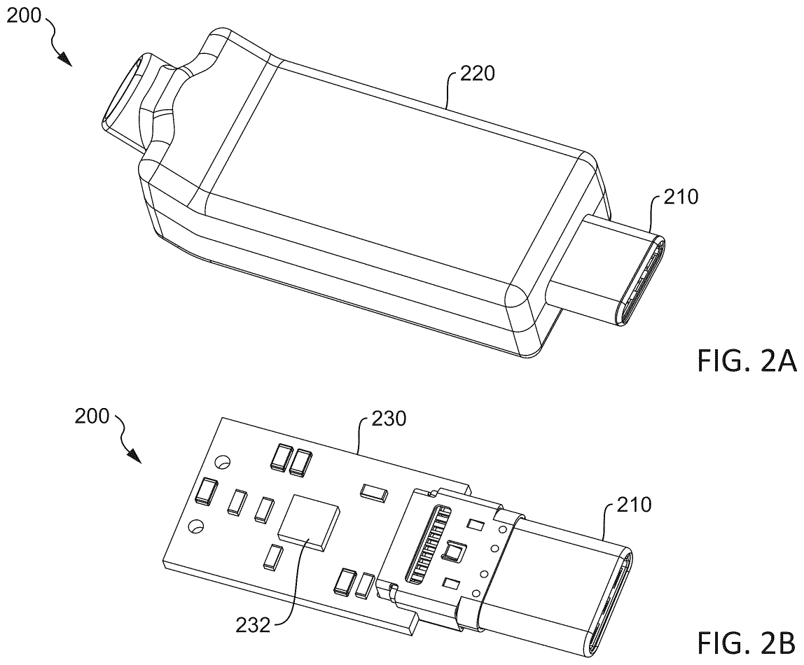

FIGS. 2A and 2B show an example connector 200 for a smart probe. FIG. 2A shows an external view, while FIG. 2B shows a view of internal components. The connector 200 includes a plug 210 that engages the socket 120. For example, the plug 210 may be a male USB-C plug. The plug 210 is communicatively coupled to a circuit board 230. The circuit board 230 includes a memory 232 that stores calibration information for the smart probe. For example, the calibration information may be written to the memory 232 during an individual calibration process for the smart probe. The circuit board 230 is surrounded by a case 220, which may include registration features for alignment with a housing of the monitoring device 100.

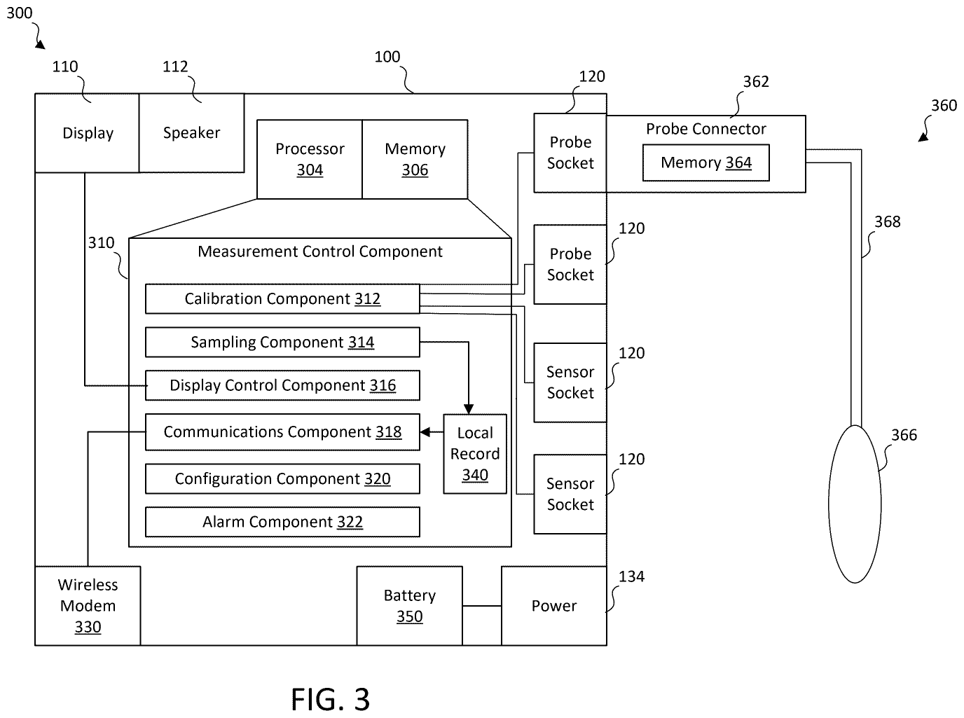

FIG. 3 is a block diagram 300 of an example monitoring device 100. As discussed above, the monitoring device 100 includes the display 110, the sockets 120, and the power port 134. The power port supplies power from a power source to a battery 350. At least one smart probe 360 is connected to one of the probe sockets 120. The smart probe 360 includes a probe connector 362 including a memory 364. The smart probe further includes a probe 366 that generates an input signal and a wire 368 that connects the probe 366 to the probe connector 362. For example, the probe 366 may be one of: a temperature probe; a temperature and humidity probe; a door sensor; a liquid level sensor; a differential pressure sensor; a liquid flow sensor; a carbon dioxide sensor; a methane sensor; or a generic voltage or current input. In some implementations, the probe 366 may have different form factors. For example, a temperature probe may be in the form of a bottle probe, a bullet probe, a vacuum bottle probe, or a plastic bottle probe.

The monitoring device 100 may include one or more processors 304 coupled to one or more memories 306. The memories 306, individually or in combination, store computer-executable instructions defining a measurement control component 310 configured to manage operation of monitoring device 100. The one or more processors 304, individually or in combination, execute the computer-executable instructions to perform the functions of the measurement control component 310 described herein.

In an aspect, the measurement control component 310 includes a calibration component 312, a sampling component 314, a display control component 316, a communications component 318. In some implementations, the measurement control component 310 optionally includes a configuration component 320 and/or an alarm component 322.

The calibration component 312 is configured to read, from the memory 364 of the smart probe 360 connected to the socket 120 of the monitoring device 100, calibration information for the smart probe; receive an input signal from the smart probe, the input signal indicative of a current measurement; and determine a value of the current measurement based on the input signal and the calibration information. In some implementations, the calibration information is a map from values of the input signal of the smart probe to values of the current measurement. The calibration information may be specific for an individual smart probe 360. For example, the calibration information may be stored in the memory 364 during an individual calibration process for the smart probe 360. The calibration information may account for variations over a range of measurement values. For example, an input signal generated by the probe 366 may not scale perfectly over the range of measurement values. The calibration information may adjust for variations over the range of measurement values such that the measurement value is calibrated within a threshold of a standard across the entire range of measurement values. For instance, for a temperature probe, the calibration information may result in a maximum temperature uncertainty of 0.024° C.

The calibration component 312 may determine a value of the current measurement by mapping a value of the input signal to the measurement value using the calibration information. For instance, the input signal may be an analog signal having a voltage and a current or a digital signal representing a value. The calibration component 312 may look up the value of the input signal on the map of the calibration information to determine the measurement value.

The display control component 316 is configured to display the value of the current measurement on the display 110. For example, the display control component 316 is configured to receive the output of the calibration component 312 for one or more probes. The display control component 316 is configured with a user interface including a display area for the measurement values for the one or more probes. The display control component 316 may generate an output image including the current measurement for output to the display 110. In some implementations, the user interface may include other information related to the current measurement such as an acceptable range for the current measurement (e.g., as defined by one or more threshold values). The display control component 316 may also indicate whether any alarms have been generated, for example, based on an indication from the alarm component 322.

The sampling component 314 is configured to periodically sample the current measurement to a local record 340. For example, the sampling component 314 may be configured with a sample period. The sample period may be selected, for example, based on a procedure or regulation. For instance, the sample period may be 5 minutes to create a record that a controlled condition has not deviated outside of a defined range. The sampling component 314 may read the current measurement (e.g., as output by the calibration component 312) and write the value of the current measurement to the local record 340. The sampling component 314 may associate the current measurement with a timestamp. In an implementation, the sampling component 314 maintains a continuous data record for a measurement when the smart probe 360 is replaced with a smart probe of the same type. That is, the sampling component 314 may sample the current measurement from the new smart probe to the same local record 340 as the previous smart probe. The calibration information ensures that the measurements are representative of a continuous series of measurements.

The communications component 318 is configured to periodically transmit a portion of the local record 340 to a remote server via a wireless network. The communications component 318 may be configured with a reporting period. The reporting period is generally greater than or equal to the sampling period. The reporting period may be selected based on remote monitoring needs of a user or organization. The communications component 318 may select a portion of the local record 340 that has not already been successfully transmitted. The communications component 318 may bundle multiple measurement values into a message for transmission to a server. In an aspect, the communications component 318 may format the message for a publish and subscribe architecture. An example of such an architecture is described in U.S. Pat. No. 10,957,182. In an implementation, the communications component 318 is further configured to connect to the server during an initial onboarding process. The communications component 318 may transmit an identification of the monitoring device 100 and receive a device template from the server.

The configuration component 320 is configured to configure the monitoring device to record and transmit probe information based on the device template. The configuration component 320 may receive the device template from the server via the communications component 318. The configuration component 320 may set a sampling period and a transmission period for one or more measurements based on the device template. In some implementations, the device template may further include alarm conditions, and the configuration component 320 may configure the alarm component 322 based on the alarm conditions.

The alarm component 322 is configured to monitor one or more alarm conditions based on the measurement values of one or more probes or sensors. For example, the alarm component 322 may be configured with an acceptable range for a current measurement defined by a lower threshold and/or an upper threshold. The alarm component 322 may generate an alarm if the current measurement exceeds a threshold (e.g., is outside of the acceptable range). In some implementations, an alarm condition may be associated with a time threshold and may only generate the alarm when the condition is satisfied for the time threshold. The time threshold may reduce false alarms. In some implementations, multiple conditions may apply to define or adjust a condition. For instance, in the case of monitoring a refrigerated unit, a door sensor may indicate a door open condition. The time threshold may be increased when a door open condition is detected to prevent a false alarm when the door is deliberately opened. The alarm component 322 may activate an alarm if the sensor detects that the refrigerated unit is opened for longer than a second time threshold. The alarm component 322 may display the alarm on the display 110. In some implementations, the monitoring device 100 includes a speaker 112 for generating an audible alarm.

In an aspect, the monitoring device 100 may operate with an external power supply or in a battery-powered mode. When operating with an external power supply, the monitoring device 100 may have no limits on transmissions or display brightness. In some implementations, in order to conserve battery power, when operating in the battery-powered mode, the monitoring device 100 may limit some features. For example, the monitoring device 100 may increase a transmission periodicity to transmit fewer messages. In some implementations, the battery-powered mode is likely to be activated when there is a power outage, so there is also a likelihood that a wireless network is unavailable when the monitoring device 100 is operating in battery-powered mode. Accordingly, less frequent transmission attempts may conserve battery power when such transmissions are more likely to fail.

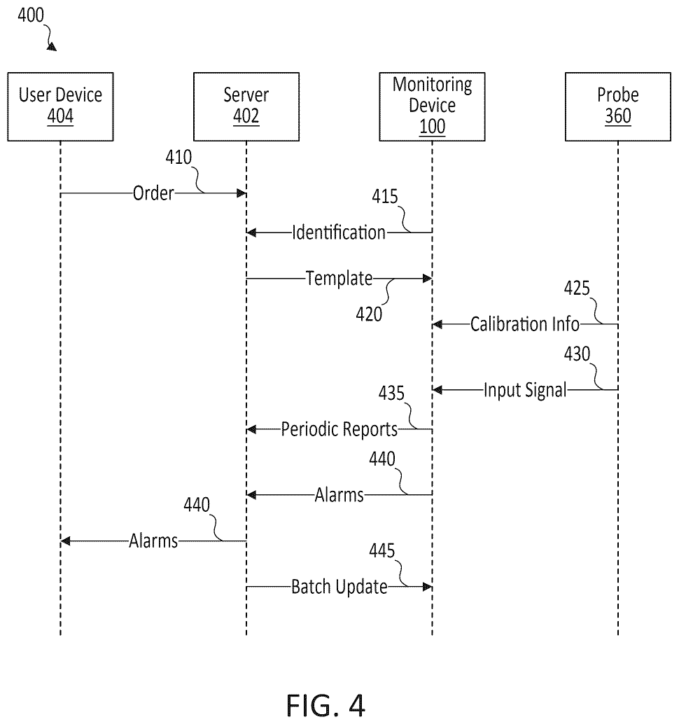

FIG. 4 is a message diagram 400 illustrating examples of messages for operating a monitoring device 100. The monitoring device 100 may communicate with a probe 360 and a server 402. The server 402 may communicate with a user device 404. In some implementations, the server 402 may be implemented on resources of a cloud service provider. The cloud service provider may instantiate one or more instances of the server 402 and route communications to the correct instance. The user device 404 may be a computing device such as a personal computer, laptop computer, tablet, or mobile device. The user device 404 may access a web application provided by the server 402. In some implementations, the user device 404 may subscribe to the server 402 according to a publish-and-subscribe architecture to receive updated records and/or alarms.

In an aspect, the user device 404 may initiate an order 410 for a monitoring device. For example, the user device 404 may submit the order 410 via a web site or web application. The order 410 may include an identification of a device template for the monitoring device 100. For instance, a user may order multiple monitoring devices for monitoring similar conditions in similar production facilities. The device template may define default configuration parameters for the device. The server 402 may associate the device template with a device identifier when the order 410 is fulfilled.

When the monitoring device 100 is powered on and connects to a wireless network, the monitoring device 100 may transmit an identification 415 of the monitoring device. The server 402 may match the identification 415 with a device template 420 based on the order 410. The server 402 may transmit the device template 420 to the monitoring device 100. The monitoring device 100 may automatically configure itself according to the device template 420. In some implementations, the monitoring device 100 may provide instructions to a technician, for example, for connecting smart probes 360 to the sockets 120.

The monitoring device 100 may read calibration information 425 from the memory 364 of the probe 360. The probe 360 may then provide an input signal to the monitoring device 100 via the socket 120. As discussed above, the monitoring device 100 may periodically record measurements based on the input signal 430 and transmit periodic reports 435 to the server 402.

In some implementations, the monitoring device 100 may transmit alarms 440 to the server 402. The server 402 may forward the alarms 440 to one or more user devices 404.

In some implementations, the monitoring device 100 may transmit a batch update 445 to one or more monitoring devices 100. The batch update 445 may be an update to the template 420. For example, the batch update 445 may be initiated by the user device 404.

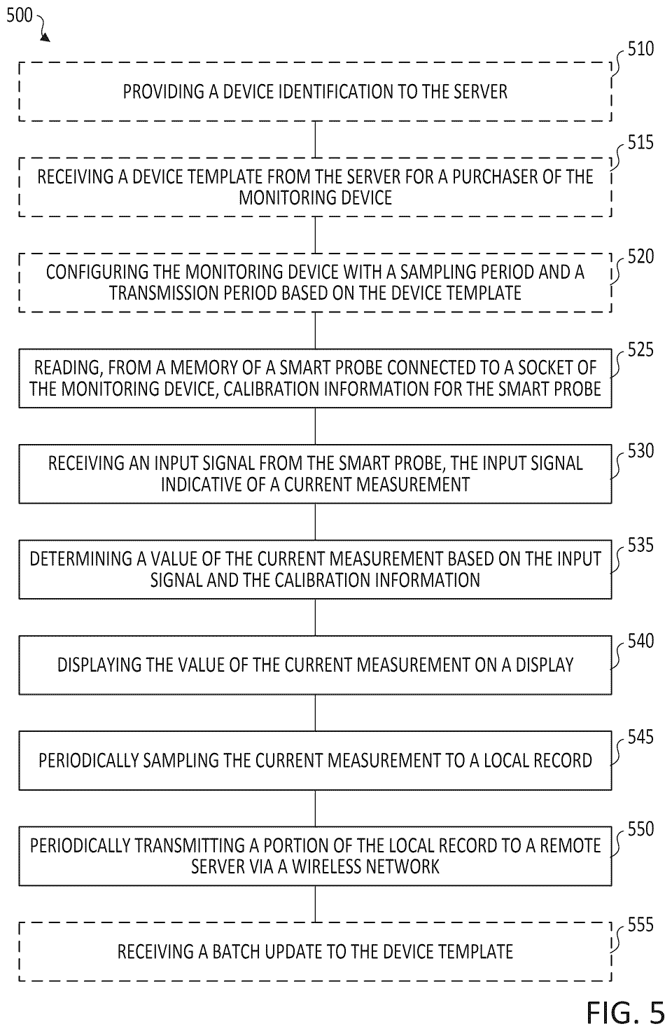

FIG. 5 is a flow diagram showing an example method 500 of operating a monitoring device 100. The method 500 may be performed by the monitoring device 100. For example, the monitoring device 100 and/or the processor 304 may execute the measurement control component 310 to perform the method 500.

In block 510, the method 500 may optionally include providing a device identification to the server. For example, the monitoring device 100 and/or processor 304 may execute the measurement control component 310 and/or the communications component 318 to provide a device identification to the server 402. For instance, the communications component 318 may transmit the device identification 415 to the server 402 via the wireless modem 330 during a device initialization process when connected to a wireless network.

In block 515, the method 500 may optionally include receiving a device template from the server for a purchaser of the monitoring device. For example, the monitoring device 100 and/or processor 304 may execute the measurement control component 310 and/or the configuration component 320 to receive a device template 420 from the server 402 for a purchaser of the monitoring device 100. For instance, the device template 420 may be based on an order 410.

In block 520, the method 500 may optionally include configuring the monitoring device with a sampling period and a transmission period based on the device template. For example, the monitoring device 100 and/or processor 304 may execute the measurement control component 310 and/or the configuration component 320 to configure the monitoring device 100 with a sampling period and a transmission period based on the device template 420.

In block 525, the method 500 includes reading, from a memory of a smart probe connected to a socket of the monitoring device, calibration information for the smart probe. For example, the monitoring device 100 and/or processor 304 may execute the measurement control component 310 and/or the calibration component 312 to read, from a memory 364 of a smart probe 360 connected to a socket 120 of the monitoring device 100, calibration information for the smart probe.

In block 530, the method 500 includes receiving an input signal from the smart probe, the input signal indicative of a current measurement. For example, the monitoring device 100 and/or processor 304 may execute the measurement control component 310 and/or the calibration component 312 to receive an input signal from the smart probe 360, the input signal indicative of a current measurement.

In block 535, the method 500 includes determining a value of the current measurement based on the input signal and the calibration information. For example, the monitoring device 100 and/or processor 304 may execute the measurement control component 310 and/or the calibration component 312 to determine a value of the current measurement based on the input signal and the calibration information.

In block 540, the method 500 includes displaying the value of the current measurement on a display. For example, the monitoring device 100 and/or processor 304 may execute the measurement control component 310 and/or the display control component 316 to display the value of the current measurement on the display 110.

In block 545, the method 500 includes periodically sampling the current measurement to a local record. For example, the monitoring device 100 and/or processor 304 may execute the measurement control component 310 and/or the sampling component 314 to periodically sample the current measurement to the local record 340.

In block 550, the method 500 includes periodically transmitting a portion of the local record to a remote server via a wireless network. For example, the monitoring device 100 and/or processor 304 may execute the measurement control component 310 and/or the communications component 318 to periodically transmit a portion of the local record to the remote server 302 via a wireless network.

In block 555, the method 500 may optionally include receiving a batch update to the device template. For example, the monitoring device 100 and/or processor 304 may execute the measurement control component 310 and/or the configuration component 320 to receive a batch update 445 to the device template 420.

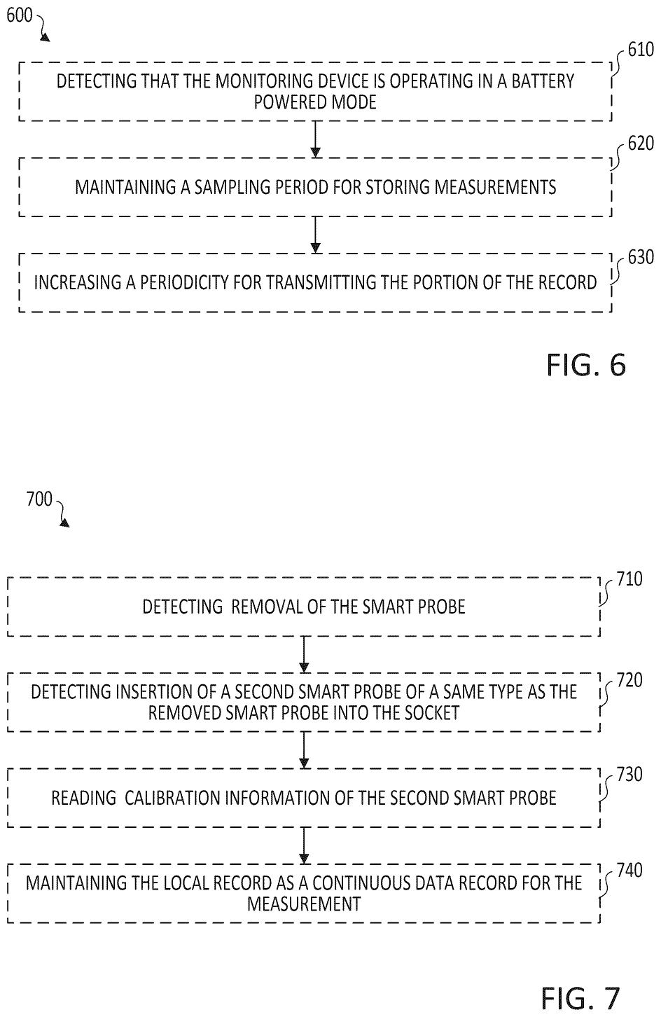

FIG. 6 is a flow diagram 600 showing additional optional actions that may be performed in conjunction with the method 500 of operating a monitoring device 100. In block 610, the method 500 may optionally include detecting that the monitoring device is operating in a battery powered mode. For example, the measurement control component 310 may detect that the battery 350 is not receiving power via the power port 134. In block 620, the method 500 may optionally include maintaining a sampling period for storing measurements. In block 630, the method 500 may optionally include increasing a periodicity for transmitting the portion of the record. Accordingly, the additional actions of flow diagram 600 may allow the monitoring device 100 to conserve battery power while maintaining a continuous record, which may be useful in the event of a power failure.

FIG. 7 is a flow diagram 700 showing additional optional actions that may be performed in conjunction with the method 500 of operating a monitoring device 100. In block 710, the method 500 may optionally include detecting removal of the smart probe. In block 720, the method 500 may optionally include detecting insertion of a second smart probe of a same type as the removed smart probe into the socket. In block 730, the method 500 may optionally include reading calibration information of the second smart probe. In block 740, the method 500 may optionally include maintaining the local record as a continuous data record for the measurement. Accordingly, the additional actions of flow diagram 600 may allow the monitoring device 100 to maintain a continuous record when a probe is replaced (e.g., for calibration purposes).

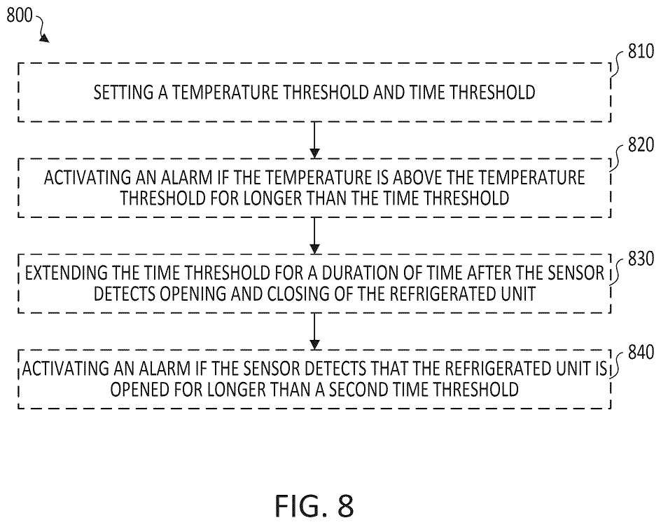

FIG. 8 is a flow diagram 800 showing additional optional actions that may be performed in conjunction with the method 500 of operating a monitoring device 100. In block 810, the method 500 may optionally include setting a temperature threshold and time threshold. In block 720, the method 500 may optionally include activating an alarm if the temperature is above the temperature threshold for longer than the time threshold. In block 730, the method 500 may optionally include extending the time threshold for a duration of time after the sensor detects opening and closing of the refrigerated unit. In block 740, the method 500 may optionally include activating an alarm if the sensor detects that the refrigerated unit is opened for longer than a second time threshold. Accordingly, the additional actions of flow diagram 800 may allow the monitoring device 100 to activate an alarm when the temperature of a controlled environment is outside of an acceptable range without generating a false alarm when the door is opened. The alarm may also be triggered when the door is inadvertently left open.

The following numbered clauses identify aspects of the disclosure:

Clause 1. A monitoring device comprising: a plurality of sockets, each socket configured to receive a smart probe including a memory storing calibration information for the smart probe; a touch screen display configured to display a current measurement based on a received input signal from the smart probe and the calibration information; and a wireless modem configured to periodically transmit one or more measurements to a remote server via a wireless network.

Clause 2. The monitoring device of clause 1, further comprising the smart probe, wherein the monitoring device is configured to copy the calibration information from the smart probe.

Clause 3. The monitoring device of clause 2, wherein the calibration information comprises a map from values of the input signal of the smart probe to values of the current measurement.

Clause 4. The monitoring device of clause 2 or 3, wherein the calibration information is stored in the memory of the smart probe based on an individual calibration of the smart probe.

Clause 5. The monitoring device of any of clauses 1-4, wherein the wireless modem is configured to transmit only measurements that have not been previously successfully transmitted.

Clause 6. The monitoring device of any of clauses 1-5, further comprising a battery, wherein the monitoring device is configured to operate in a battery powered mode in which the monitoring device maintains a sampling period for storing measurements and the wireless modem transmits the measurements at a greater periodicity.

Clause 7. The monitoring device of clauses 1-7, wherein the monitoring device is configured to maintain a continuous data record for a measurement when the smart probe is replaced with a smart probe of the same type.

Clause 8. The monitoring device of clause 1-8, wherein the smart probe is located within a refrigerated unit, wherein the monitoring device is configured to: set a temperature threshold and time threshold; and activate an alarm if the temperature is above the temperature threshold for longer than the time threshold.

Clause 9. The monitoring device of clause 8, wherein the monitoring device further comprises a socket configured to receive a sensor that detects whether the refrigerated unit is opened, wherein the monitoring device is configured to: extend the time threshold for a duration of time after the sensor detects opening and closing of the refrigerated unit; and activate an alarm if the sensor detects that the refrigerated unit is opened for longer than a second time threshold.

Clause 10. The monitoring device of any of clauses 1-9, wherein the monitoring device is configured to: provide an identification to the server; receive a device template from the server for a purchaser of the monitoring device; and configure the monitoring device to record and transmit probe information based on the device template.

Clause 11. The monitoring device of clause 10, wherein the monitoring device is further configured to receive a batch update to the device template.

Clause 12. The monitoring device of any of clauses 1-11, wherein the smart probe is one of: a temperature probe; a temperature and humidity probe; a door sensor; a liquid level sensor; a differential pressure sensor; a liquid flow sensor; a carbon dioxide sensor; a methane sensor; or a generic voltage or current input.

Clause 13. A method of operating a monitoring device, comprising: reading, from a memory of a smart probe connected to a socket of the monitoring device, calibration information for the smart probe; receiving an input signal from the smart probe, the input signal indicative of a current measurement; determining a value of the current measurement based on the input signal and the calibration information; displaying the value of the current measurement on a display; periodically sampling the current measurement to a local record; and periodically transmitting a portion of the local record to a remote server via a wireless network.

Clause 14. The method of clause 13, wherein the calibration information comprises a map from values of the input signal of the smart probe to values of the current measurement.

Clause 15. The method of clause 13 or 14, wherein the calibration information is stored in the memory of the smart probe based on an individual calibration of the smart probe.

Clause 16. The method of any of clauses 13-15, periodically transmitting a portion of the local record to a remote server comprises transmitting only measurements that have not been previously successfully transmitted.

Clause 17. The method of any of clauses 13-16, further comprising: detecting that the monitoring device is operating in a battery powered mode; maintaining a sampling period for storing measurements; and increasing a periodicity for transmitting the portion of the record.

Clause 18. The method of any of clauses 13-17, further comprising: detect removal of the smart probe; detect insertion of a second smart probe of a same type as the removed smart probe into the socket; read calibration information of the second smart probe; and maintain the local record as a continuous data record for the measurement.

Clause 19. The method of any of clauses 13-18, wherein the smart probe is located within a refrigerated unit, further comprising: setting a temperature threshold and time threshold; and activating an alarm if the temperature is above the temperature threshold for longer than the time threshold.

Clause 20. The method of clause 19, wherein the monitoring device includes a socket configured to receive a sensor that detects whether the refrigerated unit is opened, the method further comprising: extending the time threshold for a duration of time after the sensor detects opening and closing of the refrigerated unit; and activating an alarm if the sensor detects that the refrigerated unit is opened for longer than a second time threshold.

Clause 21. The method of any of clauses 13-20, further comprising: providing a device identification to the server; receiving a device template from the server for a purchaser of the monitoring device; and configuring the monitoring device with a sampling period and a transmission period based on the device template.

Clause 22. The method of clause 22, further comprising receiving a batch update to the device template.

This written description uses examples to disclose aspects of the present disclosure, including the preferred embodiments, and also to enable any person skilled in the art to practice the aspects thereof, including making and using any devices or systems and performing any incorporated methods. The patentable scope of these aspects is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. Aspects from the various embodiments described, as well as other known equivalents for each such aspect, can be mixed and matched by one of ordinary skill in the art to construct additional embodiments and techniques in accordance with principles of this application.

1. A monitoring device comprising:
a plurality of sockets, each socket configured to receive a smart probe including a memory storing calibration information for the smart probe;
a touch screen display configured to display a current measurement based on a received input signal from the smart probe and the calibration information; and
a wireless modem configured to periodically transmit one or more measurements to a remote server via a wireless network.

2. The monitoring device of claim 1, further comprising the smart probe, wherein the monitoring device is configured to copy the calibration information from the smart probe.

3. The monitoring device of claim 2, wherein the calibration information comprises a map from values of the input signal of the smart probe to values of the current measurement.

4. The monitoring device of claim 2, wherein the calibration information is stored in the memory of the smart probe based on an individual calibration of the smart probe.

5. The monitoring device of claim 1, wherein the wireless modem is configured to transmit only measurements that have not been previously successfully transmitted.

6. The monitoring device of claim 1, further comprising a battery, wherein the monitoring device is configured to operate in a battery powered mode in which the monitoring device maintains a sampling period for storing measurements and the wireless modem transmits the measurements at a greater periodicity.

7. The monitoring device of claim 1, wherein the monitoring device is configured to maintain a continuous data record for a measurement when the smart probe is replaced with a smart probe of a same type.

8. The monitoring device of claim 1, wherein the smart probe is located within a refrigerated unit, wherein the monitoring device is configured to:
set a temperature threshold and time threshold; and
activate an alarm if the temperature is above the temperature threshold for longer than the time threshold.

9. The monitoring device of claim 8, wherein the monitoring device further comprises a socket configured to receive a sensor that detects whether the refrigerated unit is opened, wherein the monitoring device is configured to:
extend the time threshold for a duration of time after the sensor detects opening and closing of the refrigerated unit; and
activate an alarm if the sensor detects that the refrigerated unit is opened for longer than a second time threshold.

10. The monitoring device of claim 1, wherein the monitoring device is configured to:
provide an identification to the server;
receive a device template from the server for a purchaser of the monitoring device; and
configure the monitoring device to record and transmit probe information based on the device template.

11. The monitoring device of claim 10, wherein the monitoring device is further configured to receive a batch update to the device template.

12. The monitoring device of claim 1, wherein the smart probe is one of:
a temperature probe;
a temperature and humidity probe;
a door sensor;
a liquid level sensor;
a differential pressure sensor;
a liquid flow sensor;
a carbon dioxide sensor;
a methane sensor; or
a generic voltage or current input.

13. A method of operating a monitoring device, comprising:
reading, from a memory of a smart probe connected to a socket of the monitoring device, calibration information for the smart probe;
receiving an input signal from the smart probe, the input signal indicative of a current measurement;
determining a value of the current measurement based on the input signal and the calibration information;
displaying the value of the current measurement on a display;
periodically sampling the current measurement to a local record; and
periodically transmitting a portion of the local record to a remote server via a wireless network.

14. The method of claim 13, wherein the calibration information comprises a map from values of the input signal of the smart probe to values of the current measurement.

15. The method of claim 13, wherein the calibration information is stored in the memory of the smart probe based on an individual calibration of the smart probe.

16. The method of claim 13, periodically transmitting a portion of the local record to a remote server comprises transmitting only measurements that have not been previously successfully transmitted.

17. The method of claim 13, further comprising:
detecting that the monitoring device is operating in a battery powered mode;
maintaining a sampling period for storing measurements; and
increasing a periodicity for transmitting the portion of the record.

18. The method of claim 13, further comprising:
detecting removal of the smart probe;
detecting insertion of a second smart probe of a same type as the removed smart probe into the socket;
reading calibration information of the second smart probe; and
maintaining the local record as a continuous data record for the measurement.

19. The method of claim 13, wherein the smart probe is located within a refrigerated unit, further comprising:
setting a temperature threshold and time threshold; and
activating an alarm if the temperature is above the temperature threshold for longer than the time threshold.

20. The method of claim 19, wherein the monitoring device includes a socket configured to receive a sensor that detects whether the refrigerated unit is opened, the method further comprising:
extending the time threshold for a duration of time after the sensor detects opening and closing of the refrigerated unit; and
activating an alarm if the sensor detects that the refrigerated unit is opened for longer than a second time threshold.

21. The method of claim 13, further comprising:
providing a device identification to the server;
receiving a device template from the server for a purchaser of the monitoring device; and
configuring the monitoring device with a sampling period and a transmission period based on the device template.

22. The method of claim 21, further comprising receiving a batch update to the device template.
